(19) United States
(12) Patent Application Publication
(10) Pub. No.: US 2022/0133512 A1
(43) Pub. Date: May 5, 2022

(54) DELIVERY DEVICES FOR EXPANDABLE IMPLANTS AND METHODS THEREFOR

(71) Applicant: Eclipse Regenesis, Inc., Menlo Park, CA (US)

(72) Inventors: James C. Y. DUNN, Palo Alto, CA (US); Andre P. BESSETTE, San Mateo, CA (US); Robert MASTON, Santa Cruz, CA (US); Carl Lance BOLING, San Jose, CA (US); Gary BOSECK, San Carlos, CA (US); Xitlalic SOTO-SIDA, Santa Clara, CA (US)

(21) Appl. No.: 17/344,368

(22) Filed: Jun. 10, 2021

Related U.S. Application Data
(60) Provisional application No. 63/037,794, filed on Jun. 11, 2020.

(51) Int. Cl. A61F 2/958

(52) U.S. Cl. CPC A61F 2/958; A61F 2002/9583

(57) ABSTRACT

Described herein are delivery devices and methods for deploying expandable implants into elongate tubular organs. The delivery devices may be configured to advance implants such as self-expanding springs within the tubular organ and release the springs at a target location where they expand and apply force to the wall of the tubular organ to lengthen the tubular organ. Delivery of the expandable implants for the treatment of small bowel syndrome is also described.

### CROSS-REFERENCE TO RELATED APPLICATIONS

This application claims priority to U.S. Provisional Application Ser. No. 63/037,794, filed on Jun. 11, 2020, the disclosure of which is hereby incorporated by reference in its entirety.

### FIELD

This application relates to delivery devices and methods for deploying expandable implants into elongate tubular organs. The implants may be self-expanding springs that may be advanced within the tubular organ in either a compressed or uncompressed state. Once implanted, the springs may expand and apply a force to the wall of the tubular organ, to thereby lengthen the tubular organ.

### BACKGROUND

Short bowel syndrome (SBS), occurs in patients with insufficient length of intestine to maintain normal digestion and absorption. SBS is a condition associated with malnutrition, malabsorption, and dehydration due to loss of large amounts of intestinal tissue. The most common causes of SBS in the pediatric population are necrotizing enterocolitis, intestinal atresias, volvulus, and abdominal wall defects.

Medical treatment for SBS includes administration of parenteral nutrition to provide necessary nutrients and hydration. Surgical treatment options for SBS include intestinal transplantation, procedures that taper and lengthen the intestine to increase absorption area, and procedures that slow down transit time, for example, colon interposition and the creation of recirculating loops. However, these procedures have had limited success and are often associated with significant complications. Hyperalimentation via the parenteral route remains the mainstay of treatment, but is often associated with complications such as catheter related infections, liver failure, and osteoporosis.

Recently, the concept of using mechanical force to lengthen intestinal tissue has been studied using a variety of tissue expander devices. Several methods of applying mechanical force to an intestinal segment have been developed, including repeated injections of saline solution, gradual advancement of a screw, and use of a hydraulic piston. However, many of these methods require repeated interventions such as serial screw advancements or saline injections. Additionally, all of these techniques incorporate a device that is at least partly outside the abdominal cavity, which introduces risks such as dislodgement, damage to the exterior component, infection, and fistula formation.

Techniques for distraction enterogenesis, where axial force is applied by one or more springs implanted in the small bowel, have also been employed to create increased intestinal length. During the distraction process, the compressed energy of the spring is slowly released into the walls of the small bowel, resulting in incremental lengthening of the tubular organ. Placement of these springs has been accomplished using open surgical procedures or described as being endoscopically delivered by deploying them with a push rod from a catheter into the intestinal tract. It would useful to have alternative ways to deliver the springs and other expandable implants into tubular organs.

### SUMMARY

Described herein are delivery devices for placing an expandable implant within an elongate tubular organ. The delivery devices may advance the expandable implants to a target location within the tubular organ in either a compressed configuration or an uncompressed configuration. When advanced in the uncompressed configuration, the implant may be compressed before release at a target location where they contact the tubular organ wall and axially expand. The implants may expand radially to engage the internal wall of a tubular organ at a target location and expand axially to lengthen the tubular organ. The axial expansion may apply a force to the organ wall capable of lengthening the tubular organ over a period of time. The expandable implants may be expandable springs or coils.

The delivery devices may be configured to place the expandable implants endoscopically, but may also be used or designed to place them within tubular organs during open surgical procedures, laparoscopy, or other minimally invasive procedures. The expandable implants are typically expandable springs, but may have any suitable structure capable of applying a longitudinal force to a tubular organ wall while allowing flow of bodily fluids therethrough. For example, braided or woven stent structures may be used. Exemplary elongate tubular organs include without limitation, the intestines (small and large), the esophagus, and blood vessels (e.g., arteries, veins, and vascular grafts).

In general, the delivery devices described herein include a shaft having a proximal end and a distal end, and a plurality of openings at the distal end of the shaft that define a seating area for the expandable implant. A retention mechanism including a plurality of filaments may also be included, where the retention mechanism is configured to secure the expandable implant to the shaft. The expandable implant may be secured to the shaft in either a compressed configuration or an uncompressed configuration. The retention mechanism may further comprise a component at the proximal end of the shaft that maintains tension on the plurality of filaments in order to keep the implant secured to the delivery device shaft. The expandable implant may be an expandable spring, as stated above. The filaments may comprise a biodegradable or non-biodegradable material, and may be a suture, ribbon, tether, or any other suitable component capable of securing the implant to the shaft. The release of filament tension against the expandable implant generally releases the implant from the delivery device.

Alternatively, the delivery devices may include a shaft having a proximal end and a distal end, and an expandable seating area at the shaft distal end for mounting the expandable implant, where the expandable implant may have a compressed configuration or an expanded configuration when mounted on or within the seating area. The expandable seating area may include a first inflatable balloon. Here the expandable implant may be mounted on the inflatable balloon in its compressed configuration such that it is circumferentially disposed about the balloon in a manner that allows the balloon to contact and provide pressure against the interior surface of the implant to secure the implant to the delivery device. After reaching the target location, the implant in its compressed state may be deployed from the delivery device by deflating the balloon. Deployment may be between two plications made in tissue at the target location. When mounted on the inflatable balloon in its uncompressed configuration, the delivery device may further include a compression mechanism to compress the implant prior to release therefrom. The compression mechanism may be a second balloon, e.g., a compression balloon, located proximal to the first balloon. In this variation, upon deflation of the first balloon, the second compression balloon may be inflated to compress the uncompressed implant between the compression balloon and a distally placed plication in tissue at the target location. Another plication may then be formed in tissue of the tubular organ proximal to the compression balloon, and the compression balloon deflated to completely release the implant from the delivery device. The compressed implant may then engage the internal wall of a tubular organ and expand axially to lengthen the tubular organ. In some instances, the expandable implant is an expandable spring.

Systems for delivering expandable implants such as springs into an elongate tubular organ are also described herein. These systems may include a loading device in which the expandable spring is housed in a compressed configuration, and a delivery device. The delivery device may include a shaft having a proximal end and a distal end, and a seating area for the expandable spring on the shaft distal end. The seating area may include an expandable component, for example, an inflatable balloon.

Methods for delivering expandable implants for tubular organ lengthening are also described herein. The methods may include: 1) mounting an expandable implant onto a delivery device, where the expandable implant may have a compressed configuration and an expanded configuration, the delivery device including a shaft having a proximal end and a distal end, a plurality of openings at the distal end of the shaft that define a seating area for the expandable implant, and a retention mechanism comprising a plurality of filaments; 2) securing the expandable implant to the seating area of the shaft by threading the plurality of filaments through the plurality of openings and applying tension to the plurality of filaments; 3) maintaining tension of the plurality of filaments during advancement of the delivery device to a target location within the elongate tubular organ; and 4) releasing the tension of the plurality of filaments when the delivery device has reached the target location to deploy the expandable implant at the target location. The implants may be mounted on the delivery device in either the compressed or uncompressed configuration. The expandable implant may be an expandable spring or coil.

Other delivery methods may include: 1) mounting an expandable implant onto a delivery device, the expandable implant having a compressed configuration and an expanded configuration, and the delivery device including a shaft having a proximal end and a distal end, and an expandable seating area at the shaft distal end for mounting the expandable implant ; 2) securing the expandable implant to the shaft by expanding the expandable seating area; 3) advancing the expandable implant to a target location within the elongate tubular organ; and 4) deploying the expandable implant at the target location by collapsing the expandable seating area. The expandable implant may be an expandable spring or coil. The expandable seating area may comprise a first inflatable balloon, and inflating the balloon may secure the implant to the seating area in either the compressed configuration or the uncompressed configuration. When mounted in the compressed configuration, the implant may be released from the delivery device between two previously sewn tissue plications at the target location. When mounted in the uncompressed configuration, the implant may be transformed to the compressed configuration by compressing the implant between a previously made distal plication and a compression mechanism of the delivery device. The compression mechanism may comprise a second balloon, e.g., a compression balloon.

In yet further methods, the delivery device may include a catheter and a push rod. Here the expandable implant may be held in its compressed state in the catheter using degradable suture. Upon advancement into a body passage of a patient, for example, the intestinal tract, using an endoscope, the expandable implant may be deployed by pushing the implant with the push rod. Thereafter, the ends of the expandable implant generally engage the interior of the body passage to maintain its position at a specific location and enable it to transfer stresses to that particular location of the intestine while the suture prevents immediate elongation of the implant. After a period of time, the degradable suture dissolves and the implant expands along the longitudinal direction thereby producing longitudinal forces in the growth direction of the intestine. The body passage may be examined periodically to check the length extension of the portion of the intestine. After a sufficient period, the expandable implant may be retracted from the body passage using an endoscope. Alternatively, the expandable implant may be left to pass naturally from the body. In some instances, the expandable implant is made from a material that degrades over a period of time.

### BRIEF DESCRIPTION OF THE DRAWINGS

FIG. 1A depicts a perspective view of an exemplary delivery device showing the seating area at the distal end of the device and a release mechanism at the proximal end of the device.

FIG. 1B shows the delivery device of FIG. 1A with an expandable spring mounted within the seating area.

FIG. 2A depicts an enlarged view of the seating area shown in FIG. 1A.

FIG. 2B depicts an enlarged view of the seating area and expandable spring shown in FIG. 1B.

FIG. 3A depicts an enlarged view of the proximal end of the delivery device shown in FIG. 1B.

FIG. 3B shows the proximal end of the delivery device of FIG. 3A with an exemplary end cap.

FIGS. 4A and 4B depict another variation of a delivery device proximal end.

FIGS. 5A and 5B depict an exemplary expandable seating area for advancing an implant in its uncompressed configuration.

FIG. 6 is a perspective view of an exemplary system including a loading device.

FIGS. 7A-7D depict an enlarged view of the proximal end of a delivery device including an exemplary ratchet gear for tensioning a plurality of filaments. FIG. 7A depicts a perspective view of the filaments attached to the ratchet gear; FIG. 7B shows a cross-sectional view of how the filaments are threaded through the delivery device shaft to attach to the ratchet; FIG. 7C depicts a side view of the proximal end of FIG. 7A; and FIG. 7D depicts a cross-sectional view of the proximal end of FIG. 7C.

### DETAILED DESCRIPTION

Described herein are delivery devices for placing an expandable implant within an elongate tubular organ. The delivery devices may be configured to deliver the implant via minimally invasive procedures, for example, using an endoscope or laparoscope, or deliver the implant during an open surgical procedure. The delivery devices may advance the expandable implant within the tubular organ in either a compressed or uncompressed configuration. When advanced in an uncompressed configuration, the delivery devices may include features for compressing the implant prior to deployment therefrom. Upon release of the expandable implant at a target location, it may contact the tubular organ wall and longitudinally (axially) expand. Some radial expansion of the implant may also occur to allow engagement with the internal wall of a tubular organ at the target location. Longitudinal expansion of the implant may apply a force to the organ wall capable of lengthening the tubular organ over a period of time. One or more implants may be delivered to achieve tubular organ lengthening. Examples of tubular organs include without limitation, the small intestine, the large intestine, the esophagus, and blood vessels. The expandable implants may be used in any instance where lengthening of a hollow organ is needed. For example, the expandable implants may be used to treat patients with short gut syndrome or esophageal atresia, or to lengthen veins or arteries prior to a grafting procedure.

### Delivery Devices

The delivery devices described herein may be configured to deliver the implant via minimally invasive procedures, for example, using an endoscope or laparoscope, or deliver the implant during an open surgical procedure. The delivery devices may advance the expandable implants within the tubular organ in a compressed configuration, and release the expandable implants at a target location where they contact the tubular organ wall and longitudinally expand. Alternatively, the delivery devices may advance the implants in an uncompressed configuration and transform the implants to a compressed configuration prior to complete release from the delivery device. Longitudinal expansion of the implant may apply a force to the organ wall capable of lengthening the tubular organ over a period of time. One or more implants may be delivered to achieve tubular organ lengthening. The delivery devices may be advanced within tubular organs such as, but not limited to, the small intestine, the large intestine, the esophagus, and blood vessels. The expandable implants may be used in any instance where lengthening of a hollow organ is needed. For example, the expandable implants may be used to treat patients with short gut syndrome or esophageal atresia, or to lengthen veins or arteries prior to a grafting procedure. In some variations, the expandable implants are expandable springs or coils.

The device for delivering an expandable implant into an elongate tubular organ may generally include a shaft having a proximal end and a distal end, and a plurality of openings at the distal end of the shaft that define a seating area for the expandable implant. The delivery device may also include a retention mechanism comprising a plurality of filaments, where the retention mechanism is configured to secure the expandable spring to the shaft and hold the expandable implant in a compressed configuration until deployment at a target location within the elongate tubular organ. However, in some instances, the plurality of filaments secure the expandable implant to the delivery device in its uncompressed configuration. In some variations, the delivery device further includes a sheath concentrically disposed about the shaft.

### Expandable Implant

The expandable implant may have any suitable structure, so long as it is capable of applying a longitudinal force to a tubular organ wall while allowing flow of bodily fluids therethrough. In one variation, the expandable implant is an expandable spring having a compressed configuration and an uncompressed (expanded) configuration. The expandable spring may be self-expanding. The expandable spring may longitudinally (axially) expand and/or longitudinally and radially expand when deployed at a target location.

In another variation, the expandable implant is a braided or woven stent. In further variations, the expandable implant is a hollow tube or cylinder, or comprises a series of linked or connected hollow tubes or cylinders. Other expandable implants that may be employed are described in U.S. Pat. No. 9,138,336 and U.S. Publication No. 2018/0333249.

The expandable implants may be made from any suitable biocompatible material or combination of materials. Selection of the material may depend on, for example, the type of implant, the intended tubular organ of deployment, duration of time estimated to achieve the desired amount of lengthening, the amount of force selected to achieve the desired amount of lengthening, or the type of anchoring desired. Exemplary materials include metals, metal alloys, biodegradable polymers, non-biodegradable polymers, shape-memory polymers, or combinations thereof. In one variation, the expandable implant is made from a metal comprising stainless steel. In another variation, the expandable implant is made from a metal alloy comprising nickel-titanium alloy (Nitinol).

Exemplary biodegradable polymers include without limitation, polyacrylates (L-tyrosine-derived or free acid), poly(a-hydroxy-esters), poly(β-hydroxy-esters), polyamides, poly(amino acid), polyalkanotes, polyalkylene alkylates, polyalkylene oxylates, polyalkylene succinates, polyanhydrides, polyanhydride esters, polyaspartimic acid, polybutylene diglycolate, poly(caprolactone), poly(caprolactone)/poly(ethylene glycol) copolymers, poly(carbonate), L-tyrosine-derived polycarbonates, polycyanoacrylates, polydihidropyrans, poly(dioxanone), poly-p-dioxanone, poly(epsilon-caprolactone), poly(epsilon-caprolactone-dimethyltrimethylene carbonate), poly(esteramide), poly(esters), aliphatic polyesters, poly(etherester), poly(ethylene glycol)/poly(orthoester) copolymers, poly(glutarunic acid), poly(glycolic acid), poly(glycolide), poly(glycolide)/poly(ethylene glycol) copolymers, poly(glycolide-trimethylene carbonate), poly(hydroxyalkanoates), poly(hydroxybutyrate), poly(hydroxybutyrate-co-valerate), poly(imino carbonates), polyketals, poly(lactic acid), poly(lactic acid-co-glycolic acid), poly(lactic acid-co-glycolic acid)/poly(ethylene glycol) copolymers, poly(lactide), poly(lactide-co-caprolactone), poly(DL-lactide-co-glycolide), poly(lactide-co-glycolide)/poly(ethylene glycol) copolymers, poly(lactide)/poly(ethylene glycol) copolymers, poly(lactide)/poly(glycolide) copolymers, polyorthoesters, poly(oxyethylene)/poly(oxypropylene) copolymers, polypeptides, polyphosphazenes, polyphosphoesters, polyphosphoester urethanes, poly(propylene fumarate-co-ethylene glycol), poly(trimethylene carbonate), polytyrosine carbonate, polyurethane, tephaflex, terpolymer (copolymers of glycolide, lactide or dimethyltrimethylene carbonate), and combinations, mixtures, or copolymers thereof. In one variation, the biodegradable polymer is poly(caprolactone) (PCL).

Examples of non-biodegradable polymers suitable to make the axially expanding implants described herein include, but are not limited to, poly(ethylene vinyl acetate), poly(vinyl acetate), silicone polymers, polyurethanes, copolymers of poly(ethylene glycol) and poly(butylene terephthalate), polystyrenes, polyvinyl chloride, polyvinyl fluoride, poly(vinyl imidazole), chlorosulphonated polyolefins, polyethylene oxide, and copolymers and blends thereof.

The implants are typically formed as self-expanding structures. When configured as a spring, the spring may be capable of multi-fold (e.g., 2-10 times) longitudinal expansion from a compressed state to an expanded state. The spring may include a plurality of coils wound to have a diameter sized to substantially match the internal diameter of the lumen of the elongate tubular organ. The gauge, pitch, and diameter of the spring may be sized to vary the force applied by it. For example, the spring may have a diameter sized for optimal engagement with the internal walls of the lumen while in its expanded form. To achieve the appropriate lengthening (distension) force, the gauge and/or pitch of the spring may be increased to increase the force applied by a spring of a set diameter. Spring diameters may range from about 0.5 cm to about 6.0 cm. For example, the spring diameter may be about 0.5 cm, about 1.0 cm, about 1.5 cm, about 2.0 cm, about 2.5 cm, about 3.0 cm, about 3.5 cm, about 4.0 cm, about 4.5 cm, about 5.0 cm, about 5.5 cm, or about 6.0 cm.

The fully expanded length (uncompressed length) of the implant, e.g., a spring, may also be configured to provide the desired level of lengthening of the tubular organ upon expansion. Implant lengths in the expanded configuration may range from about 2.0 cm to about 8.0 cm, or from about 5.0 cm to about 8.0 cm. For example, the implant length in the expanded configuration may be about 2.0 cm, about 2.5 cm, about 3.0 cm, about 3.5 cm, about 4.0 cm, about 4.5 cm, about 5.0 cm, about 5.5 cm, about 6.0 cm, about 6.5 cm, about 7.0 cm, about 7.5 cm, or about 8.0 cm. In some variations, the implant may have a length greater than 8.0 cm in the expanded configuration. In one variation, the uncompressed implant length is about 7.5 cm.

When configured as a spring, the axially expanding implant generally has a spring constant ranging from about 1.6 N/m to about 50 N/m, or from about 5 N/m to about 50 N/m. For example, the spring constant may be about 1.6 N/m. about 5 N/m, about 10 N/m, about 15 N/m, about 20 N/m, about 25 N/m, about 30 N/m, about 35 N/m, about 40 N/m, about 45 N/m, or about 50 N/m.

Anchoring of the expandable implants within the tubular organs may occur in various ways. In some variations, the ends of the implant may expand to a larger diameter than the implant body to secure it within the tubular organ. In other variations, the entire implant or portions thereof may include barbs, hooks, or stubs, or other texturing to aid in maintaining the position of the implant within the tubular organ. These anchoring members made be formed from biodegradable or non-biodegradable materials. Examples of anchoring that may be included with the implants disclosed herein are described in U.S. Pat. No. 9,138,336 and U.S. Publication No. 2018/0333249.

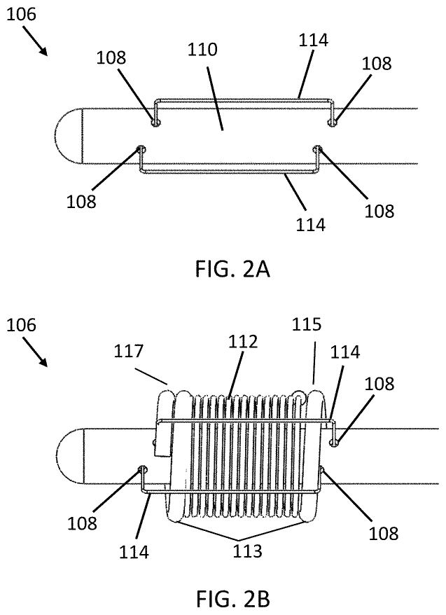

In one variation, the axially expanding implant comprises a spring having a covering, cap, or bumper on its proximal and distal ends. The covering, cap, or bumper may be made from a polymer such as silicone or polyurethane, and may protect tissue from catching on the ends of the spring and being injured, in addition to providing a larger diameter at the ends of the spring to improve the friction fit of the spring against the wall of the tubular organ. Furthermore, the covering, cap, or bumpers may improve the safety of the implant by distributing the expansion force over a larger surface area, thus reducing the stress placed on the tissue in contact with the implant. For example, the spring (112) in FIG. 2B includes bumpers (113), one at the proximal spring end (115) and one at the distal spring end (117).

In some variations, the expandable implant is an expandable spring having polyurethane bumpers at opposed ends. In one variation, the expandable spring has an uncompressed length of about 75 mm (7.5 cm). In another variation, the expandable spring has an uncompressed length of about 75 mm, a diameter of about 10 mm (1.0 cm), and a spring constant of about 0.0052 N/mm. In a further variation, the expandable spring has an uncompressed length of about 75 mm (7.5 cm), a diameter of about 14 mm (1.4 cm), and a spring constant of about 0.0074 N/mm. In yet a further variation, the expandable spring has an uncompressed length of about 75 mm (7.5 cm), a diameter of about 20 mm (2.0 cm), and a spring constant of about 0.0116 N/mm.

### Delivery Device Shaft

The shaft of the delivery device generally includes a proximal end and a distal end, and a lumen extending therebetween. The expandable implant is typically disposed at the shaft distal end within a seating area, as further described below. Mechanisms that help secure the expandable implant to the shaft during advancement within a tubular organ, as well as mechanisms that deploy the expandable implants at a target location within the tubular organ may be disposed at the proximal end, as also explained further below. The distal end may be closed, and the tip of the shaft may be rounded to prevent trauma to the tubular organ during shaft advancement.

The shaft may be made from any suitable biocompatible material. In general, the material is a biocompatible polymer such as a polyether block amide (PEBAX® elastomer). Other suitable biocompatible polymers include, but are not limited to polypropylene, polyethylene terephthalate, polyurethane, and silicone.

The length of the shaft may vary depending on such factors as the intended tubular organ of deployment, the length of the expandable implant, mode of delivery, and the age of the patient. Shaft length may range from about 20 cm to about 100 cm. For example, the shaft length may be about 20 cm, about 25 cm, about 30 cm, about 35 cm, about 40 cm, about 45 cm, about 50 cm, about 55 cm, about 60 cm, about 65 cm, about 70 cm, about 75 cm, about 80 cm, about 85 cm, about 90 cm, about 95 cm, or about 100 cm. In one variation, the shaft length is about 40 cm.

The diameter of the shaft may also vary depending on such factors as the intended tubular organ of deployment and mode of delivery. Shaft diameter may range from about 0.5 cm to about 3.0 cm. For example, the shaft diameter may be about 0.5 cm, about 1.0 cm, about 1.5 cm, about 2.0 cm, about 2.5 cm, or about 3.0 cm. In some variations, the shaft diameter is about 0.5 cm.

Variations of the delivery device having a shaft with a length of about 40 cm and a diameter of about 0.5 cm may be useful.

The shaft may also be configured to have the rigidity needed for advancement within a tubular organ as well as the flexibility that minimizes the risk perforation. In some instances, portions or sections of the shaft may be made to be more flexible or more rigid than other portions or sections. For example, the proximal end of the shaft may be formed to be more rigid than the distal end.

One or more markers may be disposed on the shaft to aid in advancement and/or visualization of the expandable implant during deployment. In some variations, the shaft may include a lubricious coating to aid in advancement of the shaft. Exemplary lubricious coatings may comprise hydrophilic polymers or polymers such as polyurethane, polyvinylpyrrolidone (PVP), or silicone.

### Seating Area

The delivery devices generally include a seating area, which is the location on the shaft where the expandable implant is secured during advancement of the delivery device within the tubular organ. If more than one implant is to be loaded on the same delivery device, the shaft will include more than one seating area. In general, the number of seating areas on the shaft will correlate to the number of implants being delivered. Implants may be mounted on or within the seating area in a compressed configuration or an uncompressed configuration.

In some variations, the shaft includes a plurality of openings at the distal end that define a seating area for the expandable implant. Any suitable number of openings may be provided at the distal end to create a seating area. Additionally, the openings may be spaced on the distal end according to any suitable arrangement. For example, the number of openings may range from two to eight for each seating area. For example, the number of openings may be two, four, or eight for each seating area. In one variation, the shaft includes eight openings arranged as four pairs at the shaft distal end. The four pairs of openings may be circumferentially arranged about the shaft distal end. In some variations, pairs of openings may be symmetrically spaced apart on the shaft. In other variations, pairs of openings may be asymmetrically spaced apart on the shaft. The openings themselves may be spaced apart from each other in any suitable amount. Spacing of the seating area from the distal tip of the shaft may range from about 1.0 cm to about 2.0 cm. In one variation, the seating area is positioned about 1.5 cm proximal to the distal tip of the shaft.

The diameter of the openings will generally be sized such that filaments of a retention mechanism may pass therethrough. The opening diameter may range from about 0.20 mm to about 0.35 mm. For example, the opening diameter may be about 0.20 mm, about 0.25 mm, about 0.30 mm, or about 0.35 mm. In one variation, the opening diameter is about 0.28 mm. Although the shape of the openings will usually be circular, they may have any shape. For example, the openings may be semi-circular, rectangular, square, or triangular.

In other variations, the seating area is expandable. The expandable seating area may include an inflatable balloon. The balloon may be compliant, semi-compliant, or non-compliant, and may have any shape suitable to help seat or secure the implant to the delivery device.

The expandable implant in its compressed or axially expanded (uncompressed) state may be circumferentially disposed about the balloon in a manner that allows the balloon to contact and provide pressure against the interior surface of the implant to secure the implant to the delivery device. The balloon may be entirely or partially inflated to help secure the implant in its axially expanded configuration to the delivery device during advancement within a tubular organ. For example, the balloon may be inflated to about 2 atm, about 4 atm, about 6 atm, about 8 atm, about 10 atm, or about 12 atm.

After reaching the target location, the expandable seating area may be collapsed, e.g., the balloon may deflated to deploy the implant from the delivery device to the target location. In variations where the implant is mounted on the expandable seating area and advanced to the target location in its compressed configuration, the compressed implant may be released from the delivery device between two previously formed tissue plications. In variations where the implant is mounted on the expandable seating area and advanced to the target location in its uncompressed configuration, the implant will generally undergo compression prior to complete deployment from the delivery device. In this variation, a compression balloon may be provided on the shaft of the delivery device proximal to the expandable seating area. After reaching the target location, the expandable seating area with the uncompressed implant may be collapsed and the compression balloon inflated to compress the implant against a first plication made in tissue at the target location that is distal to the compressed implant. After a second plication is made in the tubular organ proximal to the expandable seating area, the compression balloon may be deflated to release the compressed implant from the delivery device, and between the first and second plications.

Inflatable balloons may provide a seating area that ranges in length from about 2.0 cm to about 8.0 cm. For example, the seating area may have a length of about 2.0 cm, about 2.5 cm, about 3.0 cm, about 3.5 cm, about 4.0 cm, about 4.5 cm, about 5.0 cm, about 5.5 cm, about 6.0 cm, about 6.5 cm, about 7.0 cm, about 7.5 cm, or about 8.0 cm. In some variations, the seating area ranges in length from about 5.0 cm to about 8.0 cm.

### Retention Mechanisms

The delivery devices described herein may include a retention mechanism configured to secure the expandable implant to the delivery device shaft. The retention mechanism may secure the expandable implant to the shaft in a compressed configuration or an uncompressed configuration until deployment at a target location within an elongate tubular organ. In one variation, the retention mechanism comprises a plurality of filaments. The plurality of filaments may be threaded around or through the expandable implant, e.g., an expandable spring, and through the openings that define the seating area to secure the expandable implant to the shaft. The filaments may be fixed to the proximal end of the shaft, for example, using an adhesive. Any suitable adhesive may be employed. In other variations, the retention mechanism may comprise a plurality of ribbons, tethers, wires, or the like. The delivery devices may be pre-loaded with the expandable implant secured thereto by the filaments. In variations where the expandable implant is not pre-loaded on the delivery device, a snare may be provided to assist with threading the filaments through the device.

By being fixed at one end to the delivery device, the filaments of a retention mechanism may be held in tension by a component at the proximal end of the delivery device after threading around or through the expandable implant. In addition to being threaded around or through the expandable implant, the filaments may further be wound around other portions of the delivery device or a ratchet to keep them under tension. The retention mechanism may further include a cap that covers the filaments near their free ends, and which may be removably attached to the proximal end of the shaft. Here the retention mechanism may further include a connector coupled to the cap, where the connector includes one or more exit openings therethrough for passage of the plurality of filaments.

The plurality of filaments may include two or more filaments. For example, the plurality of filaments may include two filaments, three filaments, four filaments, five filaments, six filaments, seven filaments, or eight filaments. More than eight filaments may be employed in some variations. The filaments may range in length from about 40 cm to about 200 cm. For example, the filaments may be about 40 cm, about 45 cm, about 50 cm, about 55 cm, about 60 cm, about 65 cm, about 70 cm, about 75 cm, about 80 cm, about 85 cm, about 90 cm, about 95 cm, about 100 cm, about 105 cm, about 110 cm, about 115 cm, about 120 cm, about 125 cm, about 130 cm, about 135 cm, about 140 cm, about 145 cm, about 145 cm, about 150 cm, about 155 cm, about 160 cm, about 165 cm, about 170 cm, about 175 cm, about 180 cm, about 185 cm, about 190 cm, about 195 cm, or about 200 cm. In one variation, each filament of the plurality of filaments is about 90 cm. In another variation, each filament of the plurality of filaments has the same length. In other variations, the plurality of filaments have different lengths.

Each filament of the plurality of filaments may be a suture. In one variation, the suture is a monofilament nylon suture. In another variation, the suture is a monofilament polypropylene suture. Sutures made from other materials may also be employed.

The retention mechanisms described herein generally allow the plurality of filaments to be held under tension until the expandable implant is ready for deployment from the delivery device. Accordingly, in addition to filaments, the retention mechanisms may include other components that help to maintain tension of the filaments. These components may be disposed at the proximal end of the shaft such that they are readily controlled or actuated by the user. Upon actuation, the tension of the plurality of filaments is relaxed, thereby allowing the expandable implant to uncouple from the shaft and be deployed at the target location.

In addition to a plurality of filaments, the retention mechanism may comprise a cap, as further described below, that is removably attached to the proximal end of the shaft. Here the retention mechanism may further include a connector coupled to the cap, where the connector includes one or more exit openings therethrough for passage of the plurality of filaments. In another variation, the plurality of filaments may be attached to a ratchet gear and pawl assembly at the proximal end of the delivery device shaft. The filaments may be fixed at one end to the proximal end of the shaft, threaded through the implant at the distal end of the shaft, and then secured to the ratchet gear back at the shaft proximal end. Tension of the filaments may be adjusted by rotating the ratchet gear. Tension of the filaments may be locked by engagement of the ratchet gear with a pawl, which may also be on the shaft proximal end. Disengagement from the pawl would then release the tension and allow the expandable implant to be deployed from the delivery device.

In one variation, the delivery device may include a shaft having a proximal end and a distal end, a length of about 40 cm, and a diameter of about 0.5 mm. The shaft may include a seating area comprised of eight openings circumferentially arranged as four pairs about the shaft distal end. A retention mechanism comprising four filaments made from monofilament nylon suture may be adhered to the proximal end of the shaft and threaded around an expandable implant in its compressed configuration using the openings that define the seating area, and then back to the proximal end of the shaft. The filaments may be held under tension by features provided at the proximal shaft end. More specifically, a first end of the filaments may be fixed to the proximal shaft end via a drop of epoxy resin, and near their free ends, a cap may be placed over the four filaments to keep them under tension. Removal of the cap (i.e., actuation of the release mechanism) may release filament tension to thereby uncouple the expandable implant from the shaft and deploy the implant from the delivery device.

Systems for loading expandable implants such as expandable springs onto the delivery devices are also described herein. The systems may include a loading device that comprises an outer tube and a compression tube. The outer tube may contain a pre-loaded expandable spring in its expanded (uncompressed) configuration. Placement of the expandable spring onto a delivery device may be accomplished by inserting a delivery device including a seating area comprising an expandable component into the outer tube of the loading device. The compression tube may then be advanced within the outer tube to transform the uncompressed spring to its compressed configuration. The outer tube may include a shoulder that prevents movement of the spring during advancement of the compression tube, and against which the spring is compressed. After the spring is compressed, the expandable component is expanded to secure the compressed spring thereto. The delivery device may then be removed from the loading device. To deploy the expandable spring, the expandable device is collapsed. The expandable component within the seating area may be an inflatable balloon.

Other delivery systems may include a tube that has an inner radius sized to house an expandable spring and deliver it to a target location in an elongate tubular organ. The inside radius of the tube may be sized to house the spring in a radially compressed configuration during delivery to the target location. The tube may be a catheter or the like, wherein the spring may be pushed out of the distal end of tube via a push rod. In one variation, the tube may also comprise a section of dissolvable or absorbable material that dissolves after a short period of time in the tubular organ, releasing the spring in a radially expanded configuration such that the ends of the spring engage the wall and begin to exert a tensile stress in the longitudinal axis of the tubular organ.

The devices for delivering an expandable implant into an elongate tubular organ may generally include a shaft having a proximal end and a distal end, and a plurality of openings at the distal end of the shaft that define a seating area for the expandable implant. The delivery devices may also include a retention mechanism comprising a plurality of filaments, where the retention mechanism is configured to secure the expandable spring to the shaft and hold the expandable implant in a compressed configuration until deployment at a target location within the elongate tubular organ. A release mechanism may further be included at the proximal end of the shaft. In some variations, the delivery devices further include a sheath concentrically disposed about the shaft. In other variations, the delivery devices further include a sheath concentrically disposed at least about the seating area. The expandable implant may be an expandable spring, but other expandable structures such as braided or woven stents may also be deployed using the delivery devices.

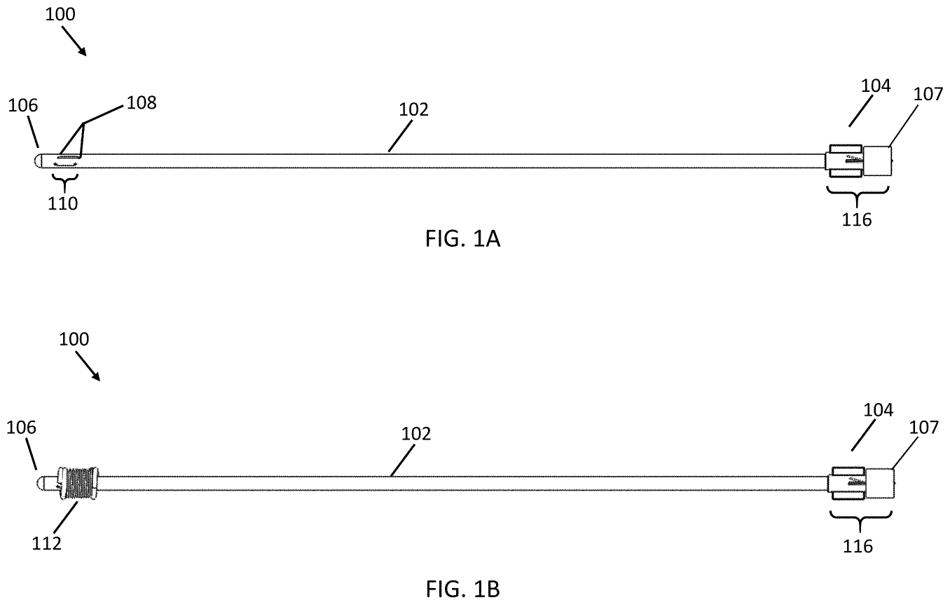

Referring to FIGS. 1A and 1B, an exemplary delivery device is shown. FIG. 1B shows an exemplary implant (an expandable spring) coupled to the delivery device. FIG. 1A provides the delivery device without the expandable spring to more clearly illustrate the seating area. In the figures, delivery device (100) includes a shaft (102) having a proximal end (104) and a distal end (106). At the distal end (106), a plurality of openings (108) are provided for passage of filaments (see elements 114 in FIG. 2A) therethrough. The plurality of openings (108) define a seating area (110) for an expandable spring (112). The plurality of filaments (114) form a part of a retention mechanism that secures the expandable spring to the shaft (102). The plurality of filaments (114) also function to hold the expandable spring (112) in a compressed configuration on the shaft (102) until deployment at a target location within an elongate tubular organ. At the proximal end (104) of the shaft (102), the retention mechanism also includes a proximal portion (116) that comprises a cap (107). The proximal retention mechanism (116) is typically configured to hold the plurality of filaments (114) in tension during advancement of the shaft (102) to the target location.

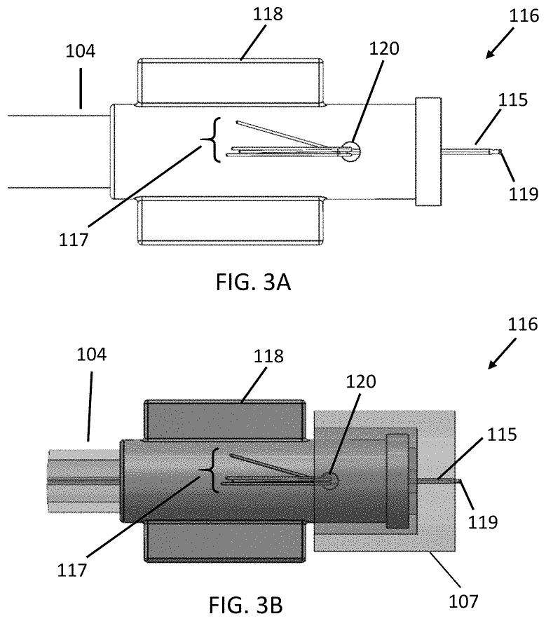

In FIGS. 2A and 2B, enlarged views of the distal end (106) of the delivery device (100) in FIGS. 1A and 1B are provided. In FIG. 2A, four openings (108) are shown at the distal end (106) of the shaft. Another four openings (not shown) are also included on the other side of the shaft, and which are configured in the same manner. The openings (108) are arranged in pairs, such that the eight openings form four pairs. The plurality of openings (108) define a seating area (110). To secure an expandable spring to the seating area (110), as illustrated in FIG. 2B, a retention mechanism including a plurality of filaments is employed. The filaments include first ends (115), which are joined to form tip (119) and free ends (117). The filaments (114) run through the shaft lumen (not shown) from the proximal end (104) of the shaft (102) toward the shaft distal end (106). The filaments (114) are then first threaded through the openings (108) closer to the shaft distal end (106) and then around the expandable spring (112) and through the openings (108) closer to the shaft proximal end (104). Given that the tip (119) (see FIGS. 3A and 3B) of the plurality of filaments (114) is attached to the cap (107) using an adhesive, pulling on the free ends (117) of the filaments (114) places them in tension so that the expandable spring (112) is secured or coupled to the shaft (102) in its compressed configuration.

The proximal retention mechanism (116) also helps to maintain tension on the filaments (114) during advancement of the delivery device. Referring to FIGS. 3A and 3B, proximal retention mechanism (116) includes a connector (118) that joins the cap (107) to the proximal end (104) of the shaft (102). When the free ends (117) are threaded through a hole (120) in the body of the connector (118), they can be held in tension by placing cap (107) over a portion of the filaments (114) exiting hole (120), as shown in FIG. 3B. The cap (107) may be removed from the connector (118) to release the tension on the filaments (114).

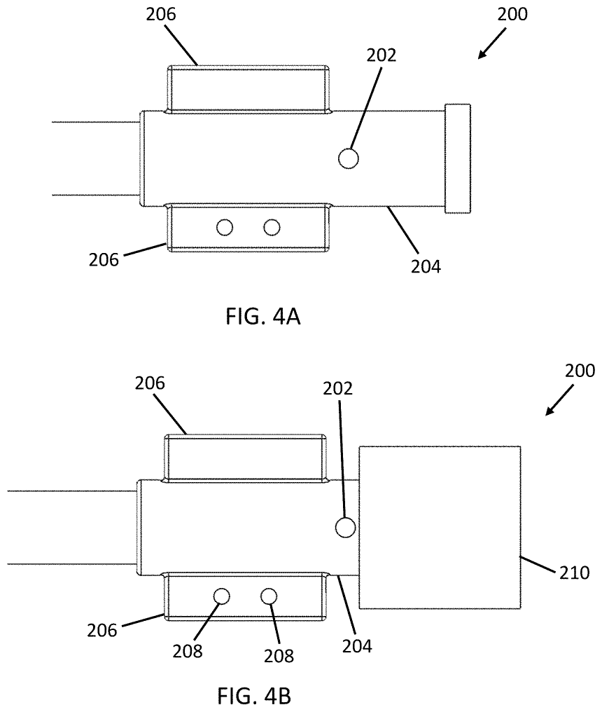

In another variation, as shown in FIGS. 4A and 4B, proximal retention mechanism (200) also includes a connector (204) having a hole (202) through which free ends of filaments may be threaded, in the same manner as described above. However, the hole (202) is positioned in the connector (204) such that cap (210) does not cover the hole (202) when coupled to the connector (204). Instead, the free ends of the filaments (not shown) are wound through wing holes (208) provided in a wing (206) of the connector (204) after exiting hole (202) and then fixed with an adhesive, for example, an epoxy resin. However, the cap (210) may be configured so that it can be pushed toward hole (202) to aid in holding tension on the filaments. The tension may be released by removing the cap (210) and cutting the filaments at the end attached to the cap (210) or the connector (204).

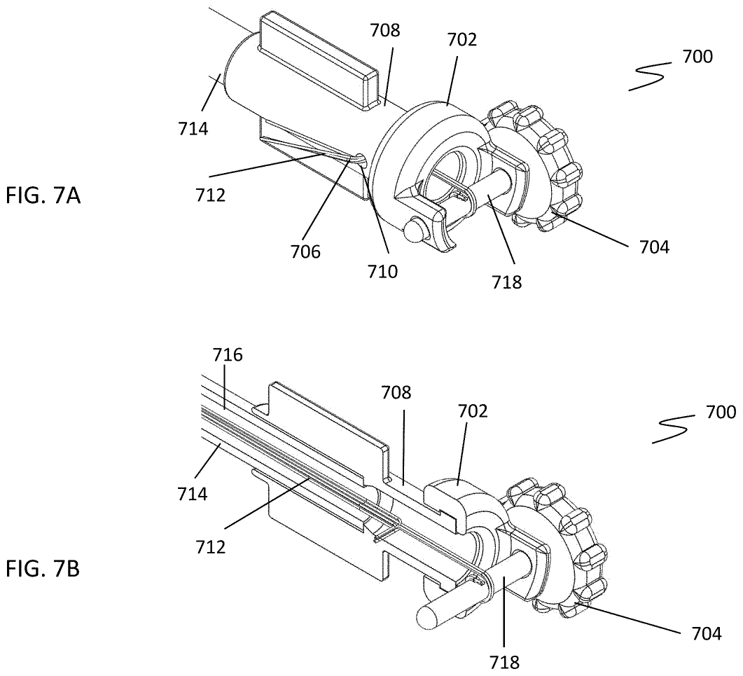

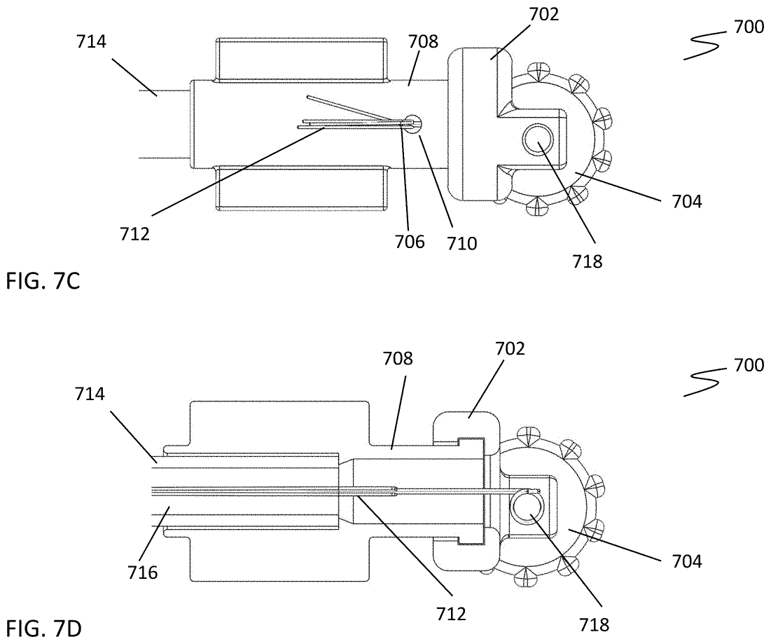

In yet another variation, the proximal retention mechanism may comprise a ratchet gear and pawl assembly. Referring to FIGS. 7A and FIG. 7C, the cap (702) of the proximal retention mechanism (700) may be modified to include a ratchet gear (704). The plurality of filaments (712) may be fixed at a point (706) on a connector (708) near hole (710). FIG. 7C shows a side view of the proximal retention mechanism (700) of FIG. 7A. In FIG. 7B, a cross-sectional view of the proximal retention mechanism (700) is provided. As shown in FIG. 7B, the plurality of filaments (712) run within the interior of the connector (708) and through the lumen (716) of the delivery device shaft (714) toward the expandable implant (not shown) and back through the connector (708) and cap (702), ending at a pin (718) coupled to the ratchet gear (704). The plurality of filaments (712) may be fixed to the pin (718) in any suitable manner, e.g., by use of an adhesive or by winding about the pin. Rotation of the ratchet gear (704) rotates the pin (718), which then tightens the filaments (712) to tension them. Rotation in the opposite direction relaxes filament tension. FIG. 7D shows a cross-sectional view of the proximal retention mechanism (700) of FIG. 7C. The ratchet gear (704) may hold tension on the filaments (712) when engaged with a pawl (not shown) to lock the position of the ratchet gear (704) and pin (718). To release filament tension, the ratchet gear (704) may be disengaged from the pawl. In variations where the filaments (712) are fixed to the pin (718) using an adhesive, the filaments (712) may be cut, and then pulled proximally to release the expandable implant from the delivery device. When an adhesive is not used and the filaments (712) are wound about the pin (718) to fix them thereto, the ratchet gear (704) may be rotated until the filaments (712) are completely unwound from the pin (718) to release the expandable implant from the delivery device.

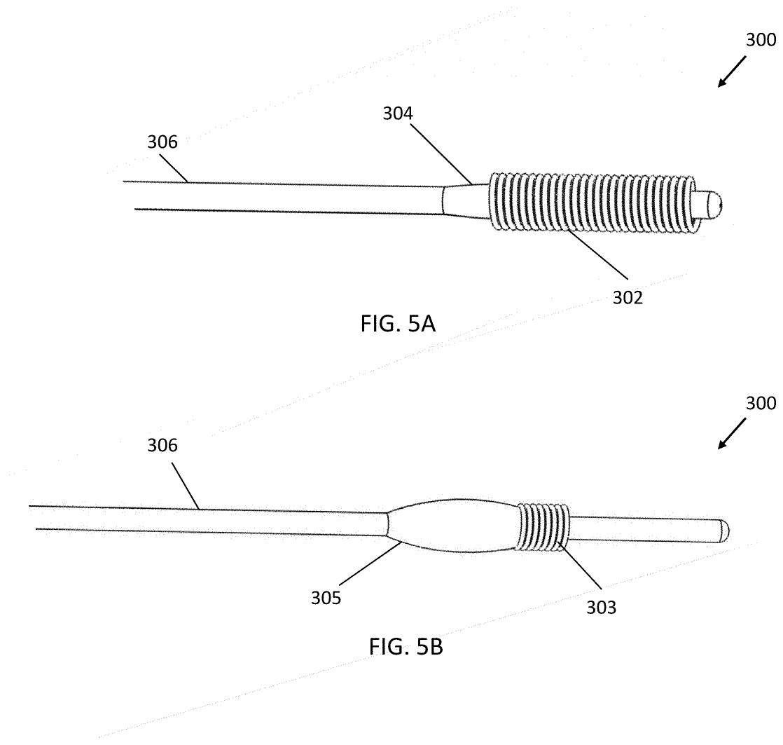

In some variations, the expandable implant may be mounted on the delivery device and advanced to a target location in an uncompressed configuration. In this variation, as shown in FIGS. 5A and 5B, the delivery device (300) may include a seating area comprising an expandable component, such as an inflatable balloon (304). Here the balloon (304) may be inflated in an amount that allows the balloon to contact and provide pressure against the interior of an expandable implant, for example, spring (302), to thereby secure the spring (302) in its uncompressed configuration to the delivery device (300). The balloon may be partially or fully inflated to secure the implant in its uncompressed configuration to the delivery device during advancement within a tubular organ. For example, the balloon may be inflated to about 2 atm, about 4 atm, about 6 atm, about 8 atm, about 10 atm, or about 12 atm. Collapse of the seating area by deflating the balloon (304) disengages the spring (302) therefrom, allowing the delivery device shaft (306) to be retracted proximally and out from the interior of the spring (302) while still maintaining the spring (302) on shaft (306). As shown in FIG. 5B, re-inflation of the balloon (304) may function as a compression mechanism when the inflated balloon is used to push the spring against a distally formed plication (not shown) to transform the spring to its compressed configuration (303). In other variations, instead of re-inflating the balloon used to seat the spring (302), a compression balloon (e.g., a second balloon) disposed proximal to the seating area may be inflated and used to push the spring against a distally formed plication to transform the spring to its compressed configuration (303). The second compression balloon may have any suitable shape. In one variation, the compression balloon may be shaped as a toroid or doughnut. After a proximal tissue plication is formed, the compression balloon may be deflated and the shaft (306) completely withdrawn from the spring (303) to deploy the spring (303) between the plications.

In further variations, the expandable implant may be mounted on the delivery device and advanced to a target location in a compressed configuration. The compressed spring may be mounted and advanced in a manner similar to that described for the uncompressed spring, however spring deployment is between two previously formed tissue plications. Thus, upon collapse of the seating area by deflation of the balloon, the compressed spring is released between the plications.

In FIGS. 5A and 5B, the length of the seating area provided by the inflatable balloon (302) may ranges from about 2.0 cm to about 8.0 cm. For example, the inflatable balloon may provide a seating area length of about 2.0 cm, about 2.5 cm, about 3.0 cm, about 3.5 cm, about 4.0 cm, about 4.5 cm, about 5.0 cm, about 5.5 cm, about 6.0 cm, about 6.5 cm, about 7.0 cm, about 7.5 cm, or about 8.0 cm. In some variations, the inflatable balloon may provide a seating area ranging in length from about 5.0 cm to about 8.0 cm.

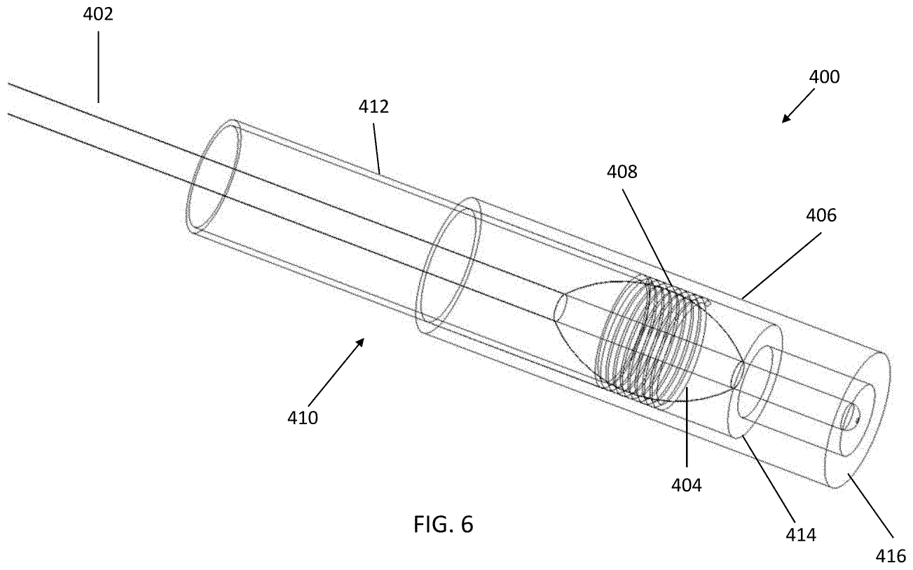

Systems for loading expandable implants such as expandable springs onto the delivery devices are also described herein. Referring to FIG. 6, the system (400) may include a loading device (410) that comprises an outer tube (406) and a compression tube (412). The outer tube (406) may be concentrically disposed about at least a portion of the compression tube (412) and may also include a shoulder (414). The outer tube (406) may contain a pre-loaded expandable spring (408) in its expanded (uncompressed) configuration. Placement of the expandable spring (408) onto delivery device (402) may be accomplished by inserting delivery device (402) including a seating area comprising an inflatable balloon (404) into the outer tube (406) of the loading device (410). The compression tube (412) may then be advanced within the outer tube (406) to transform the uncompressed spring to its compressed configuration. The shoulder (414) of the outer tube (406) prevents movement of the spring (408) during distal advancement of the compression tube (412) towards the closed end (416) of the loading device (410), such that the spring (408) is compressed against the shoulder (414). After the spring (408) is compressed, the balloon (404) is inflated to secure the compressed spring thereto. The delivery device (402) may then be removed from the loading device (410). To deploy the expandable spring (408), the balloon (404) is deflated.

Although the expandable implant is shown as a spring in the figures, it is understood that the expandable implant is not so limited, and that other expandable implants may be deployed with the delivery devices. For example, the expandable implant may be a self-expanding stent, or a braided or woven stent. Furthermore, the delivery devices may be packaged with an expandable implant pre-loaded on its distal end. Alternatively, an expandable implant may be loaded onto the delivery devices during or just prior to the start of a procedure.

### Methods

Methods for delivering expandable implants to a target location within an elongate tubular organ are also described herein. The methods may include advancing the expandable implants within the tubular organ in a compressed configuration or an uncompressed configuration, and releasing the expandable implants at a target location where they contact the tubular organ wall and longitudinally or axially expand. When advanced in the uncompressed configuration, the implants may be compressed before deployment at the target location.

The implants may expand radially to engage the internal wall of a tubular organ at a target location and expand axially to lengthen the tubular organ. The axial expansion may apply a force to the organ wall capable of lengthening the tubular organ over a period of time. The period of time may range from about one week to about 8 weeks, or from about one week to about three weeks. For example, the period of time may be about one week, about two weeks, about three weeks, about four weeks, about five weeks, about six weeks, about seven weeks, or about eight weeks.

The expandable implants may be delivered via an endoscope, a laparoscope, or during other minimally invasive procedures. In some instances, the implants may be delivered during open surgical procedures. The expandable implants are typically expandable springs, but may have any suitable structure capable of applying a longitudinal force to a tubular organ wall while allowing flow of bodily fluids therethrough. The expandable implants may be delivered to various elongate tubular organs including, but not limited to, the intestines (small and large), the esophagus, a ureter, and blood vessels (e.g., arteries, veins, and vascular grafts). In some variations, the expandable implants may be delivered or deployed between plications (e.g., between a first plication and a second plication) made in tissue or the wall of the tubular organ. The expandable implants may be used in any instance where lengthening of a hollow organ is needed. For example, the expandable implants may be used to treat patients with short gut syndrome or esophageal atresia, or to lengthen veins or arteries prior to a grafting procedure.

In one variation, the method for delivering an expandable implant into an elongate tubular organ may include: 1) mounting an expandable implant onto a delivery device, where the expandable implant may have a compressed configuration and an expanded configuration, the delivery device including a shaft having a proximal end and a distal end, a plurality of openings at the distal end of the shaft that define a seating area for the expandable implant, and a retention mechanism comprising a plurality of filaments; 2) securing the expandable implant to the seating area of the shaft by threading the plurality of filaments through the plurality of openings and applying tension to the plurality of filaments; 3) maintaining tension of the plurality of filaments during advancement of the delivery device to a target location within the elongate tubular organ; and 4) releasing the tension of the plurality of filaments when the delivery device has reached the target location to deploy the expandable implant at the target location. The implants may be mounted on the delivery device in either the compressed or uncompressed configuration. The expandable implant may be an expandable spring or coil.

Securing the expandable implant to the seating area of the shaft may include threading the plurality of filaments through the plurality of openings. As shown in FIGS. 1A to 4B, the filaments may be threaded through the lumen of the shaft from the proximal end of the delivery device through the openings located closer to the shaft distal end, and then around the expandable implant and through the openings positioned closer to the shaft proximal end. Given that one end of each filament of the plurality of filaments is attached to a retention mechanism using an adhesive, pulling on the free ends of the filaments applies tension to them so that the expandable implant is secured or coupled to the shaft, and in some variations, may be secured to the shaft in its compressed configuration.

The tension on the filaments may be maintained by mechanisms at the proximal end of the device. For example, tension may be maintained by a proximally disposed cap (107), as shown in FIG. 3B, or other covering configured to cover the tensioned filaments near their free ends. Alternatively, the filaments may be wound around other component of the device at its proximal end. For example, the filaments may be wound using wing holes (208) of a connector (204) at the proximal end a delivery device, as shown in FIG. 4B. Or, as shown in FIGS. 7A-7D, the plurality of filaments (712) may be tensioned by rotating a ratchet gear (704) provided on a cap (702), which is removably attached to the delivery device shaft (714) via connector (708).

Release of filament tension may deploy the expandable implant at the target location, and may be accomplished by removing the cap from the proximal end of the shaft, cutting the wound filaments, or rotating the ratchet gear, as previously described above.

Other delivery methods may include: 1) mounting an expandable implant onto a delivery device, the expandable implant having a compressed configuration and an expanded configuration, and the delivery device including a shaft having a proximal end and a distal end, and an expandable seating area at the shaft distal end for mounting the expandable implant; 2) securing the expandable implant to the shaft by expanding the expandable seating area; 3) advancing the expandable implant to a target location within the elongate tubular organ; and 4) deploying the expandable implant at the target location by collapsing the expandable seating area. The expandable implant may be an expandable spring or coil. The expandable seating area may comprise a first inflatable balloon, and inflating the balloon may secure the implant to the seating area in either the compressed configuration or the uncompressed configuration. When mounted in the compressed configuration, the implant may be released from the delivery device between two previously sewn tissue plications at the target location. When mounted in the uncompressed configuration, the implant may be transformed to the compressed configuration by compressing the implant between a previously made distal plication and a compression mechanism of the delivery device. The compression mechanism may comprise a second balloon, e.g., a compression balloon, as previously described above. After deployment of the expandable implant at the target location, the delivery device may be withdrawn from the elongate tubular organ.

In variations where the seating area includes an inflatable balloon, the balloons may be compliant, semi-compliant, rigid or non-compliant. The balloon may be partially or fully inflated to secure the expandable implant, e.g., an expandable spring, to the seating area. The expandable implant may be secured to the balloon in its uncompressed or compressed configuration. The balloon may be inflated to any suitable pressure that helps secure or couple the expandable implant to the delivery device.

When a compressed spring is mounted on the inflatable balloon, the device and spring may be advanced to a target location. Deflating the balloon may then release the compressed spring from the delivery device between two previously made tissue plications. Alternatively, when an uncompressed spring is mounted on the balloon and advanced, the uncompressed spring may be compressed prior to release from the delivery device. In one variation, compression may be accomplished by deflating the balloon to uncouple the uncompressed spring from the seating area, retracting the delivery device shaft until the spring is distal to the seating area, re-inflating the balloon, and then pushing the re-inflated balloon against a first plication made in tissue of an elongate tubular organ such that the spring is compressed between the balloon and the plication. A second plication may then be made, and the balloon again deflated so that the delivery device shaft may be fully withdrawn from the compressed spring to thus deploy the compressed spring between the two plications. Instead of re-inflating the balloon of the seating area, a second compression balloon may be inflated to compress the spring against the first plication, as previously described. The balloon that is part of the seating area or the compression balloon may be inflated to about 2 atm, about 4 atm, about 6 atm, about 8 atm, about 10 atm, or about 12 atm.

In yet further methods, the delivery device may include a catheter and a push rod. Here the expandable implant may be held in its compressed state in the catheter using degradable suture. Upon advancement into a body passage of a patient, for example, the intestinal tract, using an endoscope, the expandable implant may be deployed by pushing the implant with the push rod. Thereafter, the ends of the expandable implant generally engage the interior of the body passage to maintain its position at a specific location and enable it to transfer stresses to that particular location of the intestine while the suture prevents immediate elongation of the implant. After a period of time, the degradable suture dissolves and the implant expands along the longitudinal direction thereby producing longitudinal forces in the growth direction of the intestine. The body passage may be examined periodically to check the length extension of the portion of the intestine. After a sufficient period, the expandable implant may be retracted from the body passage using an endoscope. Alternatively, the expandable implant may be left to pass naturally from the body. In some instances, the expandable implant is made from a material that degrades over a period of time.

Elongate tubular organs may be lengthened by delivering one or more axially expanding implants into a tubular organ of interest. When multiple implants are placed, they may be delivered using a single delivery device. For example, multiple implants may be coupled to the same delivery device, or the delivery device may be advanced multiple times to a target location, each time deploying a single implant. In some variations, multiple implants are delivered using multiple delivery devices.

The number of implants placed in the elongate tubular organ may depend on such factors as the organ of implantation, the desired amount of lengthening, or the type of implant being employed. In general, between one and five axially expanding implants may be placed. For example, one expanding implant, two expanding implants, three expanding implants, four expanding implants, or five expanding implants may be placed at a target location in an elongate tubular organ. In one variation, at least three axially expanding implants are placed in an elongate tubular organ. In other variations, more than 5 axially expanding implants are placed in an elongate tubular organ. In further variations, multiple axially expanding implants are placed adjacent to each other in series. In some variations, multiple axially expanding implants are spaced or distributed evenly or unevenly within the elongate tubular organ. Again, placement of the implants may be via endoscopy or laparoscopy, or by an open surgical procedure. The elongate tubular organ may be the small intestine, large intestine, ureter, etc.

Although the foregoing invention has, for the purposes of clarity and understanding been described in some detail by way of illustration and example, it will be apparent that certain changes and modifications can be practiced, and are intended to fall within the scope of the appended claims.

1. A device for delivering an expandable implant into an elongate tubular organ comprising:
a shaft having a proximal end and a distal end;
a plurality of openings at the distal end of the shaft that define a seating area for the expandable implant; and
a retention mechanism comprising a plurality of filaments at the proximal end of the shaft, the retention mechanism configured to secure the expandable implant to the shaft until deployment at a target location within the elongate tubular organ.

2. The device of claim 1, wherein the expandable implant comprises a spring.

3. The device of claim 1, wherein the plurality of filaments are configured to thread around the expandable implant and through the plurality of openings to secure the expandable implant to the shaft and hold the expandable implant in a compressed configuration.

4. The device of claim 1, wherein the plurality of filaments are configured to thread around the expandable implant and through the plurality of openings to secure the expandable implant in an uncompressed configuration to the shaft.

5. The device of claim 1, wherein the retention mechanism is further configured to hold the plurality of filaments in tension during advancement of the shaft to the target location.

6. The device of claim 5, wherein the retention mechanism comprises a cap removably attached to the proximal end of the shaft.

7. (canceled)

8. The device of claim 5, wherein the retention mechanism comprises a ratchet gear and pawl assembly.

9. (canceled)

10. The device of claim 1, wherein the shaft comprises eight (8) openings arranged as four (4) pairs at the distal end.

11.-14. (canceled)

15. The device of claim 1, further comprising an expandable implant secured to the seating area.

16. The device of claim 1, wherein the device further comprises a sheath concentrically disposed about the shaft.

17. A method for delivering an expandable implant into an elongate tubular organ comprising:
mounting the expandable implant onto a delivery device, the expandable implant having a compressed configuration and an uncompressed configuration, and the delivery device comprising:
a shaft having a proximal end and a distal end;
a plurality of openings at the distal end of the shaft that define a seating area for the expandable implant; and
a retention mechanism comprising a plurality of filaments;
securing the expandable implant to the seating area of the shaft by threading the plurality of filaments through the plurality of openings and applying tension to the plurality of filaments;
maintaining tension of the plurality of filaments during advancement of the delivery device to a target location within the elongate tubular organ;
releasing the tension of the plurality of filaments when the delivery device has reached the target location; and
deploying the expandable implant at the target location.

18. The method of claim 17, wherein applying tension to the plurality of filaments comprises coupling the plurality of filaments to a cap at the proximal end of the shaft.

19. (canceled)

20. The method of claim 17, wherein applying tension to the plurality of filaments comprises coupling the plurality of filaments to a ratchet gear of a ratchet and pawl assembly.

21. (canceled)

22. The method of claim 17, wherein the expandable implant is mounted on the delivery device in the compressed configuration.

23. The method of claim 17, wherein the expandable implant is mounted on the delivery device in the uncompressed configuration.

24. The method of claim 23, further comprising inflating a balloon to transform the expandable implant to the compressed configuration.

25. The method of claim 24, wherein the balloon is inflated against plicated tissue at the target location.

26. The method of claim 17, further comprising creating at least a first plication in tissue at the target location.

27. The method of claim 17, wherein the expandable implant comprises a spring.

28. A device for delivering an expandable implant into an elongate tubular organ comprising:
a shaft having a proximal end and a distal end; and
an expandable seating area for mounting the expandable implant at the shaft distal end,
wherein the expandable implant has a compressed configuration and an uncompressed configuration, and wherein the expandable seating area is configured to secure the expandable implant to the shaft in either the compressed configuration or the uncompressed configuration.

29.-48. (canceled)
